(19) United States
(12) Patent Application Publication
(10) Pub. No.: US 2003/0163349 A1
(43) Pub. Date: Aug. 28, 2003

(54) Quality rating tool for the health care industry

(75) Inventor: Samuel W. Ho, Manhattan Beach, CA (US)

Correspondence Address: JEFFER, MANGELS, BUTLER & MARMARO, LLP, 1900 AVENUE OF THE STARS, 7TH FLOOR, LOS ANGELES, CA, 90067, US

(73) Assignee: PacifiCare Health Systems, Inc.

(21) Appl. No.: 10/086,557

(22) Filed: Feb. 28, 2002

(51) Int. Cl. A61B 5/00; G06F 19/00; G01N 37/00

(52) U.S. Cl. 705/2; 600/300; 702/81

(57) ABSTRACT

A quality rating tool used for evaluating health care providers, such as medical groups or hospitals, and a method of creating a quality rating are described. A quality rating tool is organized based on medical groups and contains numerous measures that a health care consumer can use to determine a medical group best suited to the consumer's conditions and needs. The measures include safe dosing of pain killers, use of preferred antibiotics, and overuse of antibiotics in the clinical measures category and member cost pharmacy and member cost emergency room in the affordability measures category. Values in the quality rating for these measures and others are percentiles and, as such, inform the health care consumer of where a particular medical group ranked with respect to a specific measure. The quality rating tool is compiled by an organization having access to large volumes of consumer and physician data and data from numerous other sources such as hospitals and databases of pharmacy, emergency room, professional encounter claims data, as well as consumer complaint databases and survey forms.

### FIELD OF THE INVENTION

The present invention relates to methods of creating and providing a quality rating tool for evaluating service and treatment in the health care industry. In particular, it relates to methods relating to rating tools used to measure the quality of a treatment or service provided by a health care provider.

### BACKGROUND OF THE INVENTION

Consumers, employers, business coalitions, government agencies and other organizations and individuals have long sought more detailed, meaningful information about health care providers. Presently, the health care industry provides information relating to quality of service at the health plan level. Quality information, also referred to as “report cards”, at the level of health plans, fails to provide a health care consumer with sufficient information to make informed decisions regarding the consumer's health care needs. Information regarding the quality of service and treatment from a health care provider at a more granular level would allow a consumer to select from a group of health care providers better able to meet specific needs of the consumer. However, service and treatment quality information of health care providers at a more granular level would be of significant value to health care consumers as well as health care providers.

Presently, when selecting a health care provider, such as a medical group, or when deciding on a physician, consumers are typically presented with a provider listing that states the geographic location of the provider and his or her specialty areas of practice, if applicable. In many cases, this is all the information consumers have to examine when making a decision on something as important as their health care provider.

Although not widespread, there are quality rating publications which rate health care providers with respect to a limited number of features. The ratings provide some guidance to health care consumers. However, they do not fully address some important factors to consumers when selecting a health care provider, such as patient safety or the degree of out-of-pocket expenses to be paid by a consumer.

### SUMMARY OF THE PREFERRED EMBODIMENTS

A quality rating tool that allows for consumer-focused, information-driven health care decisions with regard to selecting a health care provider is described. In one aspect of the invention, the quality rating tool, having a table format made up of columns and rows (also referred to as a quality rating table), includes a listing of some category of health care provider. In a preferred embodiment, the category of health care provider is a medical group and a row in the quality rating table corresponds to one medical group. Other categories of health care providers include hospitals and individual physicians. The columns in the quality rating table are comprised of various types of measures and scores, wherein a measure is a particular aspect of service, either health-related or administrative, care, treatment, costs and other characteristics useful in determining whether a particular medical group is well suited for a particular health consumer. Measures in the quality rating tool include safe dosing of pain killers, use of preferred antibiotics, overuse of antibiotics, member cost pharmacy, and member cost emergency room. Numerous other measures representing various aspects of a health care practice may also be included in the quality rating tool.

In a preferred embodiment, the measures in the quality rating tool are grouped into categories. These categories include clinical measures, service measures, affordability, and administrative. Clinical measures may be further categorized into staying healthy, appropriate care, and patient safety categories. For example, the safe dosing of pain killers, use of preferred antibiotics, and overuse of antibiotics are clinical measures that may fall in the patient safety sub-category. In a preferred embodiment, the member cost pharmacy and member cost emergency room measures are categorized in the affordability category. The measures are grouped into categories to facilitate use by the consumer in making a more intelligent and suitable selection of a medical group. In another preferred embodiment, the measures are not categorized. The quality rating table also contains numerous scores, such as an overall score that takes into account all the measures and overall scores for measures in each category. In a preferred embodiment, a score for a measure that a health consumer sees in the report is a percentile ranking. An actual score for the measure is derived using raw numbers and calculations derived from various data sources. The medical group's percentile ranking is based on the actual score relative to the actual scores of the other medical groups. In another preferred embodiment, the actual score may be displayed in the quality rating table.

In another aspect of the present invention, a method of creating a quality rating tool is described. The quality rating creator, typically an organization having access to a large volume of health care industry related data, selects a unit of analysis. A unit of analysis is the type of entity that is measured in the quality rating. In a preferred embodiment, a unit of analysis is a medical group. In other preferred embodiments, the unit of analysis can be a hospital, a health insurance company, a health care provider (e.g., an HMO or a PPO) or a physician. The quality rating creator collects all the data necessary for deriving the actual scores for each measure. The data can come from numerous sources including physician encounters, hospital or institutional encounters, claims and pharmacy data, customer survey and satisfaction data, and several other databases and information sources. The quality rating creator then uses this data and calculates actual scores, sometimes referred to as “raw” scores, that reflect actual performance of the medical group for the particular measure. For example, to derive the actual score for the safe dosing of pain killers measure, the number of prescriptions given by doctors in the medical group in which a dosage of four grams or less of acetaminophen (APAP) per day is maintained. A percentile ranking based on the actual score of the medical group relative to all the other medical groups in the quality rating tool is then determined.

### BRIEF DESCRIPTION OF THE DRAWINGS

FIG. 1 is a diagram showing a format of a quality rating tool for use in the health care industry in accordance with one embodiment of the present invention.

FIG. 2 is a flow diagram of a method of creating a quality rating for use in the health care industry in accordance with one embodiment of the present invention.

### DETAILED DESCRIPTION OF THE PREFERRED EMBODIMENTS

A quality rating tool for measuring various aspects of the health care industry and a method of creating the quality rating tool are described in the various figures. The present invention allows health care consumers to make more informed decisions when selecting a medical group. A health care consumer typically does not have significant information available that allows her to make a suitable selection of a medical group. Generally, a medical group is comprised of a number of physicians who form a partnership, professional corporation or other association that contracts with a health insurance company or other organization to provide medical services to health care consumers. Medical groups typically have primary care physicians who can refer patients to other specialty physicians within the medical group or outside the medical group if necessary. Some medical groups contract with nearby hospitals for inpatient or outpatient care. In most instances, a consumer selects a medical group based on the geographic location of the medical (e.g., close to home or workplace) and, in fewer instances, on recommendations from others, which can be highly subjective. For example, a person recommending or criticizing a medical group is likely doing so based on the service, treatments, etc. she received with respect to her conditions and medical needs. That is, the quality of service and treatment received from doctors in a particular medical group can vary depending on the conditions or ailments of the consumer. In any case, the health care consumer does not have access to information to medical groups that has been assimilated, evaluated, and presented in such a manner that facilitates the selection of a medical group based on particular concerns and needs of the consumer.

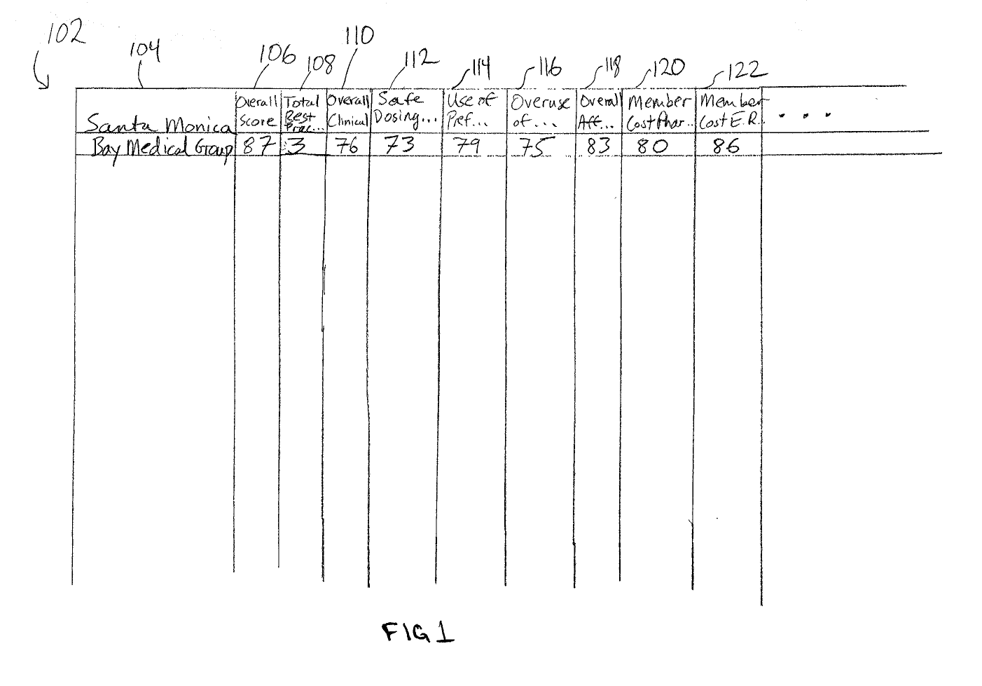

A quality rating tool of clinical, service, affordability, and administrative measures would allow a health care consumer to select a medical group most suitable for a consumer's needs. In a preferred embodiment, the measures are presented to a consumer in a format shown in FIG. 1. A quality rating table 102 has a grid structure having numerous columns and rows. A column 104 contains the name of a unit of analysis. In a preferred embodiment, the unit of analysis is a medical group. In another preferred embodiment, column 104 can contain another appropriate unit of analysis, such as physician, hospital, health insurance company, etc. In a preferred embodiment, the medical groups in column 104 are arranged based on geographic location, such as county, city, or state. In another preferred embodiment, the geographic location of the medical group or other unit of analysis can be provided in the same column or can be provided in a separate column. One aspect of medical groups generally important to a health care consumer is the ability to refer patients to an appropriate specialist or hospital. The medical group is usually responsible for authorizing a consumer's care via referrals. For example, if a consumer has a chronic condition requiring a specialist, the consumer will want to select a medical group with that specialist in its staff. With regard to hospitalization, a consumer may want to select a medical group that is contracted with a hospital close to the consumer's home. In addition to referrals, medical groups are sometimes responsible for paying claims and sending reminders about preventive care to their patients.

FIG. 1 shows a geographic indicator “Santa Monica” in column 104 and under it a medical group name “Bay Medical Group”. In a preferred embodiment, a column 106 contains an overall score of a corresponding medical group. The overall score shown for “Bay Medical Group” in FIG. 1 is 87. In a preferred embodiment the overall score for a medical group is calculated by creating a percentile ranking based on the average of all of the medical group's scores, described in greater detail below.

A column 108 indicates the number of measures in which a medical group obtained a score higher than a pre-determined value for a particular measure. A measure or measurement corresponds to a particular service or treatment. As described in greater detail below, in a preferred embodiment, a score is a percentile ranking of a medical group relative to other medical groups. In another preferred embodiment, the score can be a raw score or ‘actual’ score or any other measurement indicator. In a preferred embodiment, the column is labeled “total best practices” referring to the number of measures in which the medical group earned a “best practice” rating. A rating is a best practice rating if it is higher than the pre-determined value. For example, a percentile ranking of 90% or higher for a measure may earn a best practice standing for that measure. In FIG. 1, the medical group earned three measures having a best practice rating. It is possible that a medical group with no “Best Practice” designations may have a higher overall score than a medical group with best practices in multiple categories.

A column 110 provides an overall score for clinical measures. In a preferred embodiment, the clinical overall score is a percentile ranking based on the average of the medical group's scores of the clinical measures. In a preferred embodiment, a percentile ranking is used as the score. Thus, the overall score is an average of all the percentile rankings in the clinical, service, and affordability measures categories as well as a re-ranking based on a new scale comprised of all aggregate measures.

In a preferred embodiment, there are numerous clinical measures. The measures can be divided into sub-categories if desired. Examples of sub-categories include “Staying Healthy”, “Appropriate Care”, and “Patient Safety”. Other categories may be used when deemed appropriate. In another preferred embodiment, there may be no sub-categories of clinical measurements.

One clinical measurement is referred to in a preferred embodiment as “safe dosing of pain killers” shown as column 112. Generally, this measurement indicates the frequency with which physicians with a medical group abide by safe prescribing practices to reduce the number of preventable illnesses. In a preferred embodiment, the “safe dosing of pain killers” measurement is a percentage of the prescriptions written by doctors in the medical group in which a dosage of four grams or less of acetaminophen (APAP) per day is prescribed. A higher score or percentage for this measurement indicates a medical group having a higher percentage of prescriptions of safe dosages of APAP. That is, a higher percentage implies that doctors in the medical group generally prescribe safe doses of pain killers to patients. In another preferred embodiment, the prescriptions of another type of pain killer may be measured or the prescription of multiple pain killers may be measured. Accordingly, the dosage may vary as well depending on what dosage is considered safe for the pain killer. Regardless of the specific pain killer or pain killers reflected in the measurement, the measurement indicates the likelihood of a physician prescribing a safe dose of the pain killers.

In a preferred embodiment, the value for this measurement is derived from dividing the total number of analgesic prescriptions adjudicated for four grams or less of APAP per day by the total number of narcotic analgesic prescriptions containing APAP. As mentioned, pain killers other than APAP can be used in this measurement. The data for determining this value can be obtained from physician data and prescription claims data, typically collected by a health insurance company or any other type of health care or non-health care organization that either keeps records of prescription data or has gained access to such data.

Another measurement that may be classified as a clinical measure is referred to as “use of preferred antibiotics”. This measurement is shown in column 114. In a preferred embodiment, a value or actual score for this measurement is also a percentage ranking. The actual score is a percentage of prescribed antibiotics that are medically accepted for initiating treatment for diagnoses of bronchitis or pharyngitis. It gives an indication of the frequency with which first line antibiotics are prescribed for initiating treatment for diagnoses of bronchitis or pharyngitis. A higher percentage ranking in the quality rating indicates that physicians in the medical group are more likely to prescribe the appropriate antibiotic(s). For example, a percentile ranking of 78 means that the medical group has an actual score higher than actual scores of 77% of the other medical groups in the pool. In a preferred embodiment, the values used to derive the actual score for this measurement are the number of prescribed first line antibiotics that correlate with appropriate diagnostic tests and the number of patients with bronchitis or pharyngitis who were prescribed antibiotics. A first line antibiotic is one that is most preferably prescribed for treatment. The value is derived from dividing the number of prescribed antibiotics by the number of patients. In another preferred embodiment, antibiotics prescribed for conditions other than bronchitis and pharyngitis can be used.

A related measurement is referred to as “overuse of antibiotics” and is shown in column 116. This measure provides a percentile ranking of a medical group with respect to the number of prescribed antibiotics that coincide with appropriate diagnostic testing. A higher percentage value for this measure indicates that physicians in the medical group performed appropriate diagnostic testing when prescribing antibiotics. A higher percentage value for this measurement does not indicate that physicians in a medical group have over-prescribed antibiotics, but rather the contrary. The value is derived from dividing the number of prescribed antibiotics that correlate with appropriate diagnostic tests by the number of patients with pharyngitis who were prescribed antibiotics. In another preferred embodiment, other conditions requiring antibiotics can be used to derive the number of patients. Similar to the other measurements, the actual score is the number of prescribed antibiotics that coincide with appropriate diagnostic testing. The percentile ranking indicates the medical groups ranking with respect to the other medical groups.

Another measurement relating to clinical services measures the appropriate dispensing of flu shots to health care consumers. A flu shots measure indicates a number of health care consumers who received a flu shot for a particular medical group. Such a measure may be of particular interest to more mature health care consumers, such as those who are 65 and over. The measure is determined by dividing the number of consumers who received an influenza vaccination by the total number of consumers who were asked whether they received a vaccination. In a preferred embodiment, the data for the measure is collected from health consumer satisfaction surveys.

Another category of measures addresses costs of services and medication, which may be referred to as affordability measures. Affordability measures are chosen to indicate medical group performance in minimizing a consumer's out-of-pocket expenses in areas of pharmacy and emergency department related expenses. In a preferred embodiment, a column 118 contains an affordability overall score. Similar to the clinical overall score in column 110, the affordability score is a percentile ranking of a medical group's pharmaceutical and emergency cost controls. It measures the personal health care costs, i.e., out-of-pocket expenses, of health care consumers who are members of the medical group. The expenses relate to pharmacy and emergency department expenses. For example, a medical group with a percentile ranking of 78% means that the out-of-pocket expenses of members of the medical group were less than similar expenses of members of 77% of all other medical groups in the pool. The affordability overall score is a useful indicator for health care consumers on a strict medical budget and need to keep their personal medical expenses low.

One measurement related to affordability measures out-of-pocket expenses for pharmaceutical costs and may be referred to as “member cost pharmacy”. In a preferred embodiment, this measure is shown in column 120. In a preferred embodiment, the “member cost pharmacy” measures a patient's copayments, medications, costs for medical devices, and similar expenses not covered by the one or more health insurance organizations or other type of health care organizations compiling data for the measurements. For example, the data for the member cost pharmacy can be taken from a database of claims submitted by health care consumers relating to pharmacy claims. From these claims the insurance company or other health care organization can derive the amount not covered by the consumer's health insurance and, therefore, had to be paid by the consumer. Generally, the measurement indicates potential financial impact to a health care consumer and promotes cost-conscious prescriptions of pharmaceuticals and other health-related products.

Since health care consumers differ with respect to which areas of care and service are most important to them, the quality rating tool may provide a broad selection of measurements of performance thereby allowing the consumer to focus on the specific quality areas that are most important to the consumer.

Another measurement related to affordability addresses emergency room costs paid by the member. This measurement is shown in column 122. The measurement indicates a health care consumer's emergency medical, pharmaceutical and other costs not covered by the consumer's health insurance and had to be paid for personally. Similar to the member cost pharmacy measure, this measure demonstrates financial impact to a consumer and promotes reasonable costs among medical groups relating to emergency medical costs. Data for this measurement can be gathered from emergency room claims.

There are numerous other measures and measurement categories useful to consumers when selecting a medical group. For example, in the clinical measures category there can be measures for cervical cancer screening, breast cancer screening, eye exam for diabetics, optimal outpatient care to avoid hospitalization, prescription drug treatment for asthma, and hospital readmissions, among others. The clinical measures can further be categorized into measures for “staying healthy,” “appropriate care” and “patient safety.”

Another category of measurements is a service measures category that measures the quality of services offered by the medical group. Examples of measurements in the service measures category include member satisfaction with contracting medical group, member satisfaction with primary care physician, member satisfaction with timeliness of the referral process, primary care access complaints, and overturned appeals, among others. All these measures can be grouped in the aforementioned categories or can be presented to the health care consumer as a stand-alone measurement.

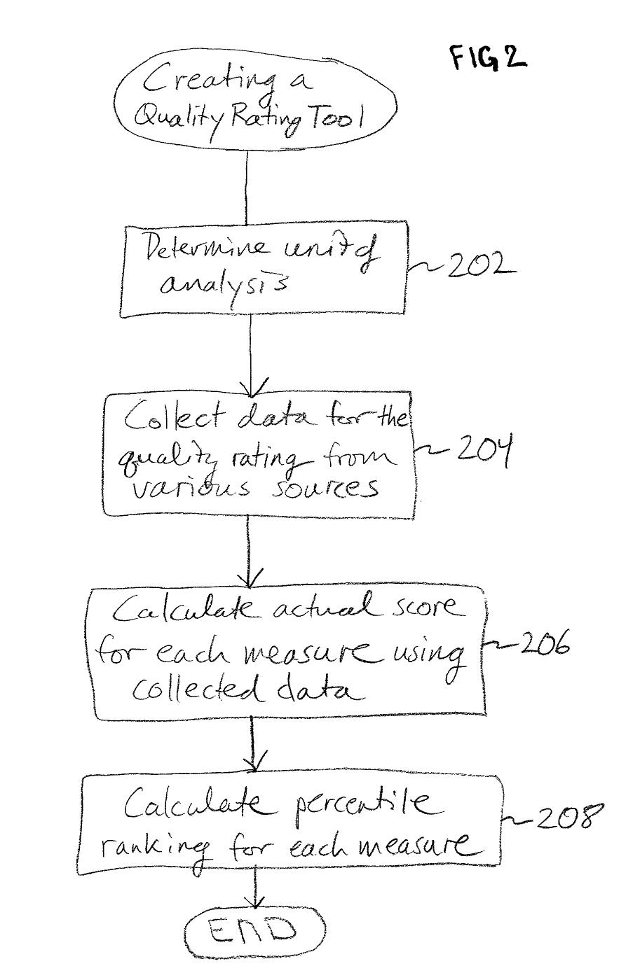

A method of creating a quality rating is described in FIG. 2. At step 202 an appropriate unit of analysis is selected by the creator of the quality rating tool. In a preferred embodiment, the unit of analysis is a medical group, as shown in column 104 of FIG. 1. Examples of other units of analysis include physicians, hospitals, health insurance companies, health care providers, and so on. Generally, a unit of analysis should have a statistically meaningful volume of data that is associated with the unit of analysis. For example, in some cases, an individual physician or health care provider may not be an appropriate unit of analysis if the physician has a small number of patients. This would cause the level of care and service measured at the physician level to be less reliable than measurements of medical groups with large numbers of patients.

At step 204 data for compiling the quality rating table is collected from various sources. One source is forms collected from physicians and medical groups. In a preferred embodiment, data for the measures, such as the clinical measures, is collected from insurance companies or health care organizations capable of collecting large volumes of health care and health industry related data. For example, the insurance companies collect the data from the physicians based on services rendered and claims/encounter data submitted by the physician. In a preferred embodiment, the data is collected from diagnostic codes and physical exam codes. Data for service measures is gathered from member satisfaction surveys, complaint databases, phone calls from consumers, and so on. In another preferred embodiment, data for the service measures is collected from physician encounter forms, pharmaceutical claim forms, emergency room claims, hospital claims, and other sources. Other sources include customer surveys and similar materials. In a preferred embodiment, the data from the various sources are stored in one database. If there is insufficient data for a particular measure, the percentile ranking or other type of score for the measure is not provided in the quality rating tool. An indicator such as “DBT” representing “data below threshold” is stated in the quality rating table in instances where there is insufficient data to assure a stable result, or a result that would otherwise be attained by random variation of occurence. At step 206 the quality rating provider calculates the actual scores for each measure for each group. The raw scores are calculated for each measure. Actual scores are derived from the various data sources and measure a particular aspect of a medical group. At step 208 the quality rating creator calculates a percentile ranking for the measure.

In a preferred embodiment, the quality rating table includes only medical provider groups having an average of at least 1,000 health care consumers during a one-year period. Data for clinical measures can be used from a two or three year period for more accurate percentile rankings. Data for some of the measures can be taken from health consumer surveys, for example, completed by customers of a particular health insurance company. The survey may present questions such as the following:

a) How often did you get the medical care or advice you needed when you phoned your doctor's office when the office was closed?

b) At those times when you felt you needed a specialist, how often was your primary care physician willing to refer you?

c) How would you rate your ability to be referred to a specialist when needed?

d) How would you rate the timeliness of the referral process?

e) Did you get a flu (influenza) shot in 2001?

f) Have you ever had a pneumonia vaccination?

g) Has your doctor inquired about all adverse drug reactions and allergies you have experienced?

h) How often did doctors or other health providers listen carefully to you?, and

i) How often did doctors or other health providers explain things in a way you could understand?

The quality rating tool has a number of uses for various audiences. In a preferred embodiment, the primary audience consists of a health care organization's members and prospective members. In many cases the health care organization is a health insurance company and the members are the insured. It is typically the health care organization that has access to a significant volume of the type of data needed to compile a quality rating tool of the present invention. Another entity, such as an online portal, a public health agency or other type of public service entity, a health insurance broker, a company offering various health plans, and so on may also offer a quality rating tool to its constituents by gaining access to the data stored by another entity or arranging use of the quality rating in some manner from an entity compiling and maintaining the quality rating table. For example, a company can get a license from a quality rating creator and make it available to the company's employees. In another example, the quality rating creator may license the quality rating to other commercial entities, such as an online portal (e.g., Yahoo, AOL), a general information health care web site, a health insurance broker, or to any other entity, including other PPOs and HMOs, finding a use for the quality rating tool.

The quality rating tool can be used to help identifying not only local health care providers that excel in particular area but also those with superior total performance scores. Another intended audience is the health care providers who can see how the medical group they practice in compares with other providers. If necessary, they can take steps to improve their services accordingly. Another audience is the employers who can add value to their benefits by offering to its employees the quality rating table before the employees select health plans, often referred to as “open enrollment” to help in their selection of a health plan. Thus, the quality rating, a snapshot of a medical group's performance in clinical and service quality, and affordability and administrative accuracy, enables a health care organization to create a more consumer-focused, information-driven health care system.

In a preferred embodiment, the quality rating creator is not recommending one medical group over another. Every medical provider group examined in the quality rating tool provides adequate and safe health care to the consumers. It is possible that a medical provider group with an excellent reputation may have no “total best practices” indicators in column 108. A medical group may excel at an aspect of health care which is not covered by the quality rating tool, since the profile represents only a sampling of measures. To obtain a “best practice” score for a particular measure, the medical provider group must deliver superior performance as well as sufficient data to reflect its performance. If data availability or accuracy is poor, scores will not necessarily reflect true performance. In a preferred embodiment, there is a wide breadth of measures provided in the quality rating tool. This is because what is important in terms of quality varies greatly for different people. A particular measure that is an important quality factor for one health care consumer may not be important to another. Thus, there should be a wide range of clinical, service, affordability, and administrative measures.

The embodiments of the present invention recited herein are intended to be merely exemplary and those skilled in the art will be able to make numerous modifications to them without departing from the spirit of the present invention. For example, the quality rating tool, and in particular, the affordability measures, can include cost factors in the equations for determining out-of-pocket expenses other than those for pharmaceuticals and emergency medical services. Other measures regarding affordability or costs can be included that are within the scope of the measures described. In another example, the patient safety measures can take into account dosing of medications other than painkillers and antibiotics and can use numerous other conditions as sample conditions. In another example, the quality rating tool may have fewer or more columns or the order of the columns may be different. The quality rating tool may contain only a subset of the measures described or may have all the measures described and additional rating or quantitative data. All such modifications are intended to be within the scope of the present invention as defined by the claims.

1. A quality rating tool comprising:
a listing of one or more entities, an entity being a type of health care provider; and
a measure indicating the frequency with which a level of dosing of a medication was prescribed by an entity, wherein the quality rating tool is used to select an entity for providing health care services to a health care consumer.

2. A quality rating tool as recited in claim 1 wherein the medication includes painkillers.

3. A quality rating tool as recited in claim 2 wherein the measure is derived from a number of prescriptions in which a predetermined amount by weight of painkillers is not exceeded.

4. A quality rating tool as recited in claim 3 wherein the predetermined dosage is four grams and the painkiller is acetaminophen (APAP).

5. A quality rating tool as recited in claim 1 wherein the level of dosing indicated is a safe level of dosing.

6. A quality rating tool as recited in claim 1 wherein an entity is a medical group.

7. A quality rating tool as recited in claim 1 wherein an entity is a hospital.

8. A quality rating tool as recited in claim 1 wherein the measure is categorized in a clinical measures category.

9. A quality rating tool as recited in claim 8 wherein the measure is further categorized in a patient safety category.

10. A quality rating tool comprising:
a listing of one or more entities, an entity being a type of health care provider; and
a measure indicating the frequency with which use of an antibiotic for treating a condition is prescribed by an entity, wherein the quality rating tool is used to select an entity for providing health care services to a health care consumer.

11. A quality rating tool as recited in claim 10 wherein the measure is an indication of a number of prescribed antibiotics where the prescribed antibiotics are medically accepted for initiating treatment of a condition.

12. A quality rating tool as recited in claim 10 wherein the condition includes bronchitis.

13. A quality rating tool as recited in claim 10 wherein the measure is derived from a number of preferred antibiotic prescriptions and a number of total antibiotic prescriptions for treating the condition.

14. A quality rating tool as recited in claim 10 wherein an entity is a medical group.

15. A quality rating tool as recited in claim 10 wherein an entity is a hospital.

16. A quality rating tool as recited in claim 10 wherein the measure is represented in the quality rating tool as a percentile ranking.

17. A quality rating tool as recited in claim 10 wherein the measure is categorized in a clinical measures category.

18. A quality rating tool as recited in claim 17 wherein the measure is further categorized in a patient safety category.

19. A quality rating tool comprising:
a listing of one or more entities, an entity being a type of health care provider; and
a measure indicating the frequency with which appropriate diagnostic testing was performed when prescribing antibiotics for a condition, wherein the quality rating tool is used to select an entity for providing health care services to a health care consumer.

20. A quality rating tool as recited in claim 19 wherein the measure is an indication of a number of prescriptions that correspond with appropriate diagnostic testing.

21. A quality rating tool as recited in claim 19 wherein the measure is derived from a number of prescribed antibiotics that correspond with appropriate diagnostic testing and a number of patients with the condition.

22. A quality rating tool as recited in claim 21 wherein the condition includes pharyngitis.

23. A quality rating tool comprising:
a listing of one or more entities, an entity being a type of health care provider; and
a measure indicating the cost of health services and products incurred by a health care consumer obtaining health care services from an entity, wherein the quality rating tool is used to select an entity for providing health care services.

24. A quality rating tool as recited in claim 23 wherein a measure indicating the cost of health services and products incurred by a health care consumer includes a measure indicating pharmaceutical costs incurred by a health care consumer.

25. A quality rating tool as recited in claim 23 wherein a measure indicating the cost of health services and products incurred by a health care consumer includes a measure indicating emergency medical costs incurred by a health care consumer.

26. A quality rating tool measuring the performance of a medical group from a plurality of medical groups, the quality rating tool comprising:
a safe dosing of a medication measure;
a use of preferred antibiotics measure; and
a overuse of antibiotics measure, wherein the quality rating tool is used by a health care consumer in selecting an appropriate medical group.

27. A quality rating tool measuring the performance of a medical group from a plurality of medical groups, the quality rating tool comprising:
a member pharmaceutical cost measure; and
a member emergency services cost measure wherein the quality rating tool is used by a health care consumer in selecting an appropriate medical group.

28. A method of creating a quality rating tool in the health care industry, the method comprising:
selecting a unit of analysis which defines the granularity of the quality rating tool;
collecting data from a plurality of sources relating to a plurality of measures used in the quality rating tool;
calculating a score for a measure from the plurality of measures using data from the plurality of sources, wherein the measures include a safe dosing of a medication measure

29. A method of creating a quality rating tool in the health care industry, the method comprising:
selecting a unit of analysis which defines the granularity of the quality rating tool;
collecting data from a plurality of sources relating to a plurality of measures used in the quality rating tool;
calculating a score for a measure from the plurality of measures using data from the plurality of sources, wherein the measures include use of preferred antibiotics measure.

30. A method of creating a quality rating tool in the health care industry, the method comprising:
selecting a unit of analysis which defines the granularity of the quality rating tool;
collecting data from a plurality of sources relating to a plurality of measures used in the quality rating tool;
calculating a score for a measure from the plurality of measures using data from the plurality of sources, wherein the measures include an overuse of antibiotics measure.
